(19) United States
(12) Patent Application Publication
(10) Pub. No.: US 2014/0212487 A1
(43) Pub. Date: Jul. 31, 2014

(54) SOLID DISPERSION FORMULATION OF AN ANTIVIRAL COMPOUND

(71) Applicant: Gilead Pharmasset LLC, Foster City, CA (US)

(72) Inventors: Erik Mogalian, San Francisco, CA (US); Reza Oliyai, Burlingame, CA (US); Dimitrios Stefanidis, Mountain View, CA (US); Vahid Zia, San Carlos, CA (US)

(21) Appl. No.: 14/168,329

(22) Filed: Jan. 30, 2014

Related U.S. Application Data
(60) Provisional application No. 61/759,310, filed on Jan. 31, 2013.
(60) Provisional application No. 61/870,721, filed on Aug. 27, 2013.

(51) Int. Cl. A61K 47/38; A61K 47/32; A61K 47/48; A61K 31/4184

(52) U.S. Cl. CPC A61K 47/38; A61K 31/4184; A61K 47/32; A61K 47/48853
USPC 424/465; 514/394; 424/482; 264/13

(57) ABSTRACT

Disclosed are solid dispersions comprising ledipasvir, wherein ledipasvir is dispersed within a polymer matrix formed by a pharmaceutically acceptable polymer, and further wherein ledipasvir is substantially amorphous. Also disclosed are pharmaceutical compositions comprising solid dispersion and methods of using the solid dispersion.

### CROSS-REFERENCE TO RELATED APPLICATION

This application claims the benefit under 35 U.S.C. §119(e) to U.S. Provisional Application No. 61/759,310, filed on Jan. 31, 2013, and U.S. Provisional Application No. 61/870,721, filed on Aug. 27, 2013, both of which are incorporated herein by reference in their entirety.

### BACKGROUND

Hepatitis C is recognized as a chronic viral disease of the liver which is characterized by liver disease. Although drugs targeting the liver are in wide use and have shown effectiveness, toxicity and other side effects have limited their usefulness Inhibitors of hepatitis C virus (HCV) are useful to limit the establishment and progression of infection by HCV as well as in diagnostic assays for HCV.

Ledipasvir (GS-5885), having the chemical name (1-{3-[6-(9,9-difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carbonyl}-2-methyl-propyl)-carbamic acid methyl ester, is known to be an effective anti-HCV agent, as described for example in WO 2010/132601 and U.S. Pat. No. 7,964,580. However, solid dispersion formulations of ledipasvir with improved pharmacokinetic properties were not heretofore known.

### SUMMARY

Ledipasvir has the chemical name (1-{3-[6-(9,9-difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carbonyl}-2-methyl-propyl)-carbamic acid methyl ester, and is a HCV NS5A inhibitor that has demonstrated potent anti-HCV activity against genotype (1a and 1b) HCV infection. Ledipasvir has the following chemical formula:

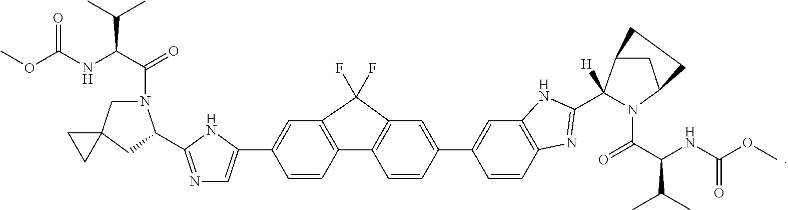

The amorphous solid dispersion formulation of ledipasvir shows unexpected benefits over other formulations of ledipasvir. For example, the amorphous solid dispersion demonstrates increased bioavailability, a reduction or elimination of food-effect, a reduction in negative drug-drug interactions with acid suppressive therapies, a reduction in variability across patient populations, and an improvement in dose linearity at higher doses.

Aspects of the disclosure relate to solid dispersions comprising ledipasvir, wherein ledipasvir is dispersed within a polymer matrix formed by a pharmaceutically acceptable polymer, and further wherein the ledipasvir is substantially amorphous.

Further aspects of the disclosure relate to pharmaceutical compositions comprising the solid dispersion and a pharmaceutically acceptable carrier, pharmaceutical dosage forms, and tablets. The disclosure also provides methods for making the solid dispersion and methods for using them in the treatment of hepatitis C virus.

### BRIEF DESCRIPTION OF THE DRAWINGS

FIG. 1 shows a comparison of dissolution profiles for ledipasvir formulations: amorphous free base of ledipasvir using a conventional formulation; 10 mg tablet employing the crystalline D-tartrate salt of ledipasvir in a convention formulation; 10 mg tablet employing the crystalline D-tartrate salt of ledipasvir in a conventional formulation; and 30 mg tablet employing an amorphous solid dispersion of ledipasvir comprising copovidone in a drug:polymer ratio of 1:1.

FIG. 2 is a XRPD pattern of the solid dispersion formulation of ledipasvir comprising copovidone in a drug:polymer ratio of 1:1. As shown by the XRPD, the solid dispersion is in the amorphous state.

FIG. 3 is a modulated differential scanning calorimetry (DSC) curve of the solid dispersion of ledipasvir comprising copovidone in a drug:polymer ratio of 1:1. The glass transition temperature of the solid dispersion is about 140° C.

FIG. 4 shows a solid state characterization of the solid dispersion formulation of ledipasvir comprising copovidone in a drug:polymer ratio of 1:1 by solid state nuclear magnetic resonance (SS-NMR).

FIG. 5 is a Fourier-transformed Raman spectra of the solid dispersion of ledipasvir comprising copovidone in a drug:polymer ratio of 1:1.

### DETAILED DESCRIPTION

### 1. Definitions

As used in the present specification, the following words and phrases are generally intended to have the meanings as set forth below, except to the extent that the context in which they are used indicates otherwise.

As used herein, the term “about” used in the context of quantitative measurements means the indicated amount ±10%. For example, “about 2:8” would mean 1.8-2.2:7.2-8.8.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point).

The term “substantially amorphous” as used herein is intended to mean that greater than 50%; or greater than 55%; or greater than 60%; or greater than 65%; or greater than 70%; or greater than 75%; or greater than 80%; or greater than 85%; or greater than 90%; or greater than 95%, or greater than 99% of the compound present in a composition is in amorphous form. “Substantially amorphous” can also refer to material which has no more than about 20% crystallinity, or no more than about 10% crystallinity, or no more than about 5% crystallinity, or no more than about 2% crystallinity.

The term “polymer matrix” as used herein is defined to mean compositions comprising one or more polymers in which the active agent is dispersed or included within the matrix.

The term “solid dispersion” refers to the dispersion of one or more active agents in a polymer matrix at solid state prepared by a variety of methods, including spray drying, the melting (fusion), solvent, or the melting-solvent method.

The term “amorphous solid dispersion” as used herein, refers to stable solid dispersions comprising an amorphous active agent and a polymer. By “amorphous active agent,” it is meant that the amorphous solid dispersion contains active agent in a substantially amorphous solid state form.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

The term “carrier” refers to a glidant, diluent, adjuvant, excipient, or vehicle with which the compound is administered. Examples of carriers are described herein and also in “Remington's Pharmaceutical Sciences” by E. W. Martin.

The term “polymer” refers to a chemical compound or mixture of compounds consisting of repeating structural units created through a process of polymerization. Suitable polymers useful in this invention are described throughout.

The term “pharmaceutically acceptable polymer” refers to a polymer that does not have properties that would cause a reasonably prudent medical practitioner to avoid administration of the material to a patient, taking into consideration the disease or conditions to be treated and the respective route of administration.

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropylcellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, and alginic acid.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, or talc; and solubilizers such as fatty acids including lauric acid, oleic acid, and C₈/C₁₀ fatty acid.

The term “film coating” refers to a thin, uniform, film on the surface of a substrate (e.g. tablet). Film coatings are particularly useful for protecting the active ingredient from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “therapeutically effective amount” refers to an amount that is sufficient to effect treatment, as defined below, when administered to a mammal in need of such treatment. The therapeutically effective amount will vary depending upon the subject being treated, the weight and age of the subject, the severity of the disease condition, the manner of administration and the like, which can readily be determined by one of ordinary skill in the art.

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “% w/w” as used herein refers to the weight of a component based on the total weight of a composition comprising the component. For example, if component A is present in an amount of 50% w/w in a 100 mg composition, component A is present in an amount of 50 mg.

### 2. Solid Dispersions of Ledipasvir

The solid dispersion as described herein demonstrate increased bioavailability, a reduction or elimination of food-effect, a reduction in negative drug-drug interactions with acid suppressive therapies, a reduction in variability across patient populations, and an improvement in dose linearity at higher doses.

The solid dispersion of ledipasvir comprises the compound in substantially an amorphous state dispersed within a polymer matrix formed by a pharmaceutically acceptable polymer. The starting material of the solid dispersion can be a variety of forms of ledipasvir including crystalline forms, amorphous form, salts thereof, solvates and free base. For example, the D-tartrate salt, anhydrous crystalline free base, amorphous free base, solvates or desolvates of ledipasvir can be used. Solvates of ledipasvir include, for example, those described in U.S. 2013/0324496 (incorporated herein by reference) such as, for example, the monoacetone solvate, diacetone solvate, ethyl acetone solvate, isopropyl acetate solvate, methyl acetate solvate, ethyl formate solvate, acetonitrile solvate, tetrahydrofuran solvate, methyl ethyl ketone solvate, tetrahydrofuran solvate, methyl ethyl ketone solvate, and methyl tent-butyl ether solvate. Particular starting material contemplated to be useful are the monoacetone solvate, diacetone solvate, anhydrous crystalline free base, D-tartrate salt, anhydrous crystalline free base, and amorphous free base. These forms are characterized and described in U.S. Patent Publication No. 2013/0324496.

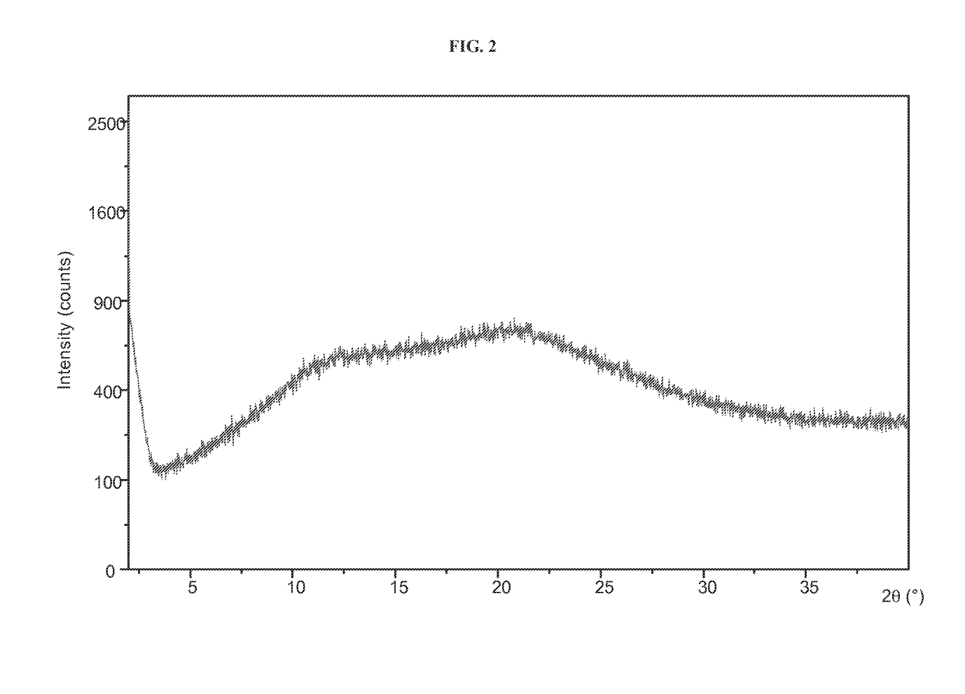

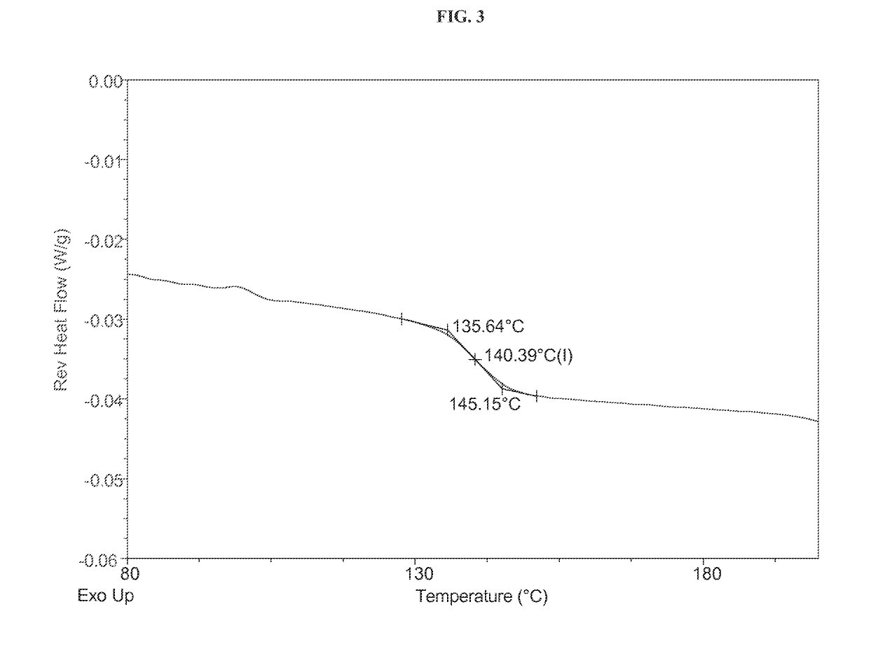

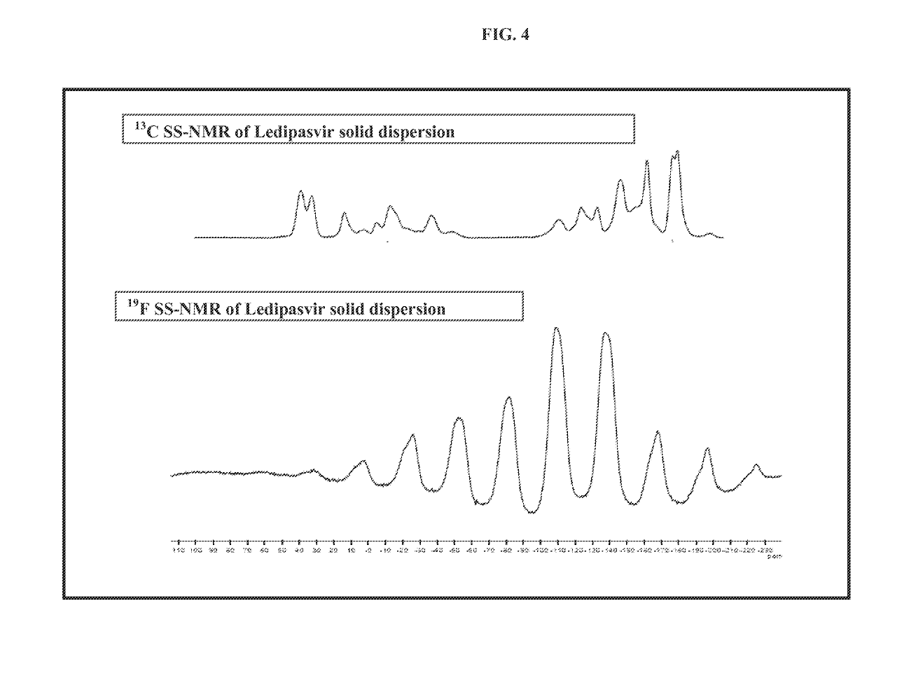

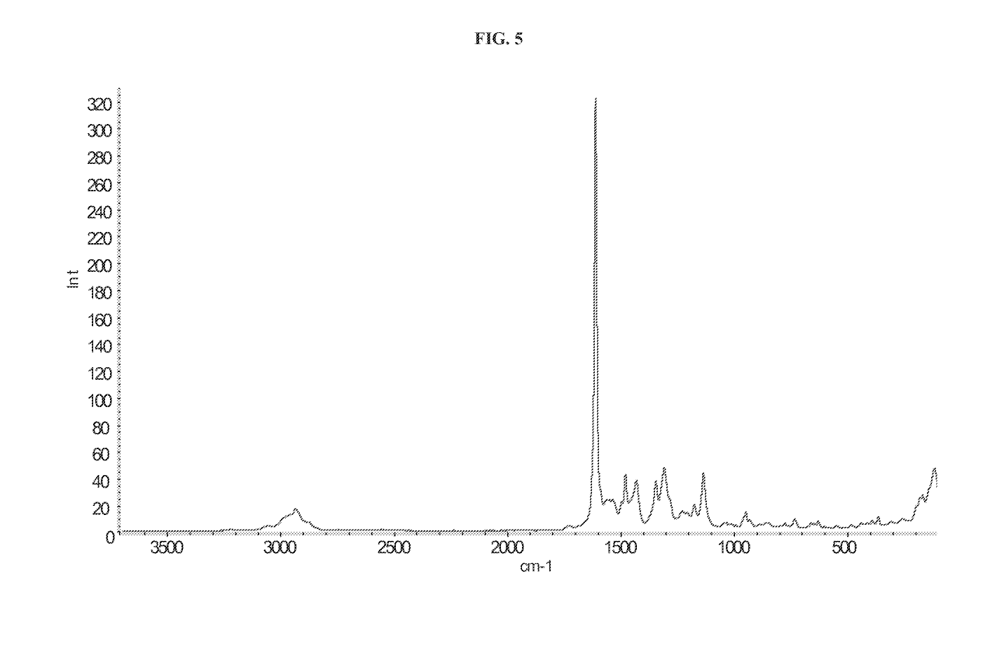

After dispersion with the polymer, the solid dispersion is in the amorphous form. FIGS. 2-5 characterize the amorphous solid dispersion comprising ledipasvir. As shown by the XRPD in FIG. 2, the solid dispersion is in the amorphous state, and the glass transition temperature of the solid dispersion comprising ledipasvir copovidone in a 1:1 drug:polymer ratio is about 140° C., as shown in FIG. 3. The amorphous solid dispersions of ledipasvir and other polymers, including hypromellose, copovidone and povidone in either a 2:1 or 1:1 drug:polymer ratio resulted in single glass transition temperature ranging from 140 to 173° C., which temperatures are at least 100° C. above the temperatures of the downstream manufacturing process, transportation/distribution, and storage. This large difference in temperature significantly reduces the potential for recrystallization of ledipasvir in the formulation matrix. The solid dispersions comprising the polymers just mentioned were stored in open condition at 40° C./75% RH for up to 4 weeks, and the physical stability of the dispersions were determined using DSC and XRPD. Regardless of polymer type, all ledipasvir:polymer 2:1 dispersions remained amorphous without an apparent phase transition or recrystallization.

In one embodiment, the polymer used in the solid dispersion of ledipasvir is hydrophilic. Non-limiting examples of hydrophilic polymers include polysaccharides, polypeptides, cellulose derivatives such as methyl cellulose, sodium carboxymethylcellulose, hydroxyethylcellulose, ethylcellulose, hydroxypropyl methylcellulose acetate-succinate, hydroxypropyl methylcellulose phthalate, cellulose acetate phthalate, and hydroxypropylcellulose, povidone, copovidone, hypromellose, pyroxylin, polyethylene oxide, polyvinyl alcohol, and methacrylic acid copolymers.

In a further embodiment, the polymer is non-ionic. Non-ionic polymers showed benefits in screening solubility experiments. Non-limiting examples of non-ionic polymers include hypromellose, copovidone, povidone, methyl cellulose, hydroxyethyl cellulose, hydroxypropyl cellulose, ethylcellulose, pyroxylin, polyethylene oxide, polyvinyl alcohol, polyethylene glycol, and polyvinyl caprolactam-polyvinyl acetate-polyethylene glycol.

In another embodiment, the polymer is ionic. Examples of ionic polymers include hydroxypropyl methylcellulose acetate-succinate, hydroxypropyl methylcellulose phthalate, cellulose acetate phthalate, and methacrylic acid copolymers.

Solubility screening experiments were performed for ledipasvir in the presence of a variety of polymers, including hydroxypropyl cellulose, hypromellose, povidone and copovidone. Aqueous solubility of ledipasvir was determined at pH 2 and pH 5 in the presence of 0.1% w/w polymer. Hypromellose, povidone, and copovidone increased the solubility of ledipasvir. At pH 5, the aqueous solubility of ledipasvir increased 3-, 4-, and 10-fold in the presence of hypromellose, povidone, and copovidone. Thus, in one embodiment, the polymer is selected from hypromellose, povidone, and copovidone.

In a further embodiment, the polymer is selected from the group consisting of hypromellose, copovidone, and povidone. Hypromellose and copovidone solid dispersions both showed adequate stability and physical characteristics. As shown in Table 5 in Example 4, a copovidone-based dispersion increased bioavailability more than the equivalent hypromellose-based formulation (F=30% and 22%, respectively) when prepared at 2:1 ledipasvir:polymer ratio. Accordingly, in a specific embodiment, the polymer is copovidone.

In certain embodiments, the weight ratio of ledipasvir to polymer is from about 5:1 to about 1:5. Throughout the disclosure the ratio of ledipasvir to polymer may be expressed as API:polymer or drug:polymer. In further embodiments, the weight ratio of ledipasvir to polymer is about 5:1 to about 1:4, or from about 5:1 to about 1:3, or from about 5:1 to about 1:2, or from about 2:1 to about 1:2, or from about 2:1 to about 1:1. In a specific embodiment, the weight ratio of ledipasvir to polymer is about 1:1. In another embodiment, the weight ratio of ledipasvir to polymer is about 2:1. In further embodiments, the weight ratio of ledipasvir to polymer is about 5:1, 1:4, 1:3, or 1:2. Increasing the fraction of polymer to a 1:1 ratio may, in some instances, result in an increased bioavailability. For example, Table 5 in Example 4 demonstrates that a 1:1 ratio of ledipasvir:copovidone resulted in increased bioavailability (F=35%) in famotidine pretreated dogs.

### 3. Pharmaceutical Compositions for Oral Delivery

The solid dispersions of ledipasvir provided in accordance with the present disclosure are usually administered orally. This disclosure therefore provides pharmaceutical compositions that comprise a solid dispersion comprising ledipasvir as described herein and one or more pharmaceutically acceptable excipients or carriers including but not limited to, inert solid diluents and fillers, diluents, including sterile aqueous solution and various organic solvents, permeation enhancers, solubilizers, disintegrants, lubricants, binders, glidants, adjuvants, and combinations thereof. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The active ingredient (i.e., solid dispersion of ledipasvir) may be present in the pharmaceutical composition in a therapeutically effective amount. In some embodiments, the pharmaceutical compositions comprises from about 1% to about 80% w/w of the solid dispersion of ledipasvir. In further embodiments, the composition comprises from about 5% to about 75% w/w, or from about 5% to about 60% w/w, or from about 5% to about 55% w/w, or from about 5% to about 50% w/w, or from about 5% to about 45% w/w, or from about 5% to about 40% w/w, or from about 5% to about 35% w/w, or from about 5% to about 30% w/w, or from about 5% to about 25% w/w, or from about 5% to about 20% w/w, or from about 10% to about 75% w/w, or from about 10% to about 60% w/w, or from about 10% to about 55% w/w, or from about 10% to about 50% w/w, or from about 10% to about 45% w/w, or from about 10% to about 40% w/w, or from about 10% to about 35% w/w, or from about 10% to about 30% w/w, or from about 10% to about 25% w/w, or from about 10% to about 20% w/w, or from about 20 to about 40% w/w of the solid dispersion of ledipasvir. In a specific embodiment, the pharmaceutical composition comprises about 18% w/w of the solid dispersion of ledipasvir. In a further specific embodiment, the pharmaceutical composition comprises about 30% of the solid dispersion of ledipasvir. In further embodiments, the pharmaceutical composition comprises about 5% w/w, about 10% w/w, about 20% w/w, about 25% w/w, about 30% w/w, about 35% w/w, about 40% w/w, or about 45% w/w of the solid dispersion of ledipasvir.

Ledipasvir may be present in the pharmaceutical composition in a therapeutically effective amount. In some embodiments, the pharmaceutical compositions comprises from about 1% to about 50% w/w of ledipasvir. In further embodiments, the composition comprises from about 1% to about 40% w/w, or from about 1% to about 30% w/w, or from about 5% to about 25% w/w, or from about 10% to about 20% w/w, or from about 13% to about 17% w/w of ledipasvir. In further embodiments, the pharmaceutical composition comprises about 5% w/w, about 7% w/w, about 10% w/w, about 12% w/w, about 18% w/w, about 20% w/w, about 25% w/w, about 30% w/w, about 35% w/w, about 40% w/w, about 45% w/w, or about 50% w/w of ledipasvir. In a specific embodiment, the pharmaceutical composition comprises about 15% w/w of ledipasvir.

In one embodiment, the pharmaceutical composition comprises about 15 to about 30% w/w of a solid dispersion comprising substantially amorphous ledipasvir dispersed within a polymer matrix formed by a pharmaceutically acceptable polymer, wherein the weight ratio of ledipasvir to polymer is from about 2:1 to about 1:2.

The pharmaceutical compositions may be administered in either single or multiple doses by oral administration. Administration may be via capsule, tablet or the like. In one embodiment, the compound is in the form of a tablet. In a further embodiment, the tablet is a compressed tablet. In making the pharmaceutical compositions that include the solid dispersion described herein, the active ingredient is usually diluted by an excipient and/or enclosed within such a carrier that can be in the form of a capsule, tablet, sachet, paper or other container. When the excipient serves as a diluent, it can be in the form of a solid, semi-solid or liquid material (as above), which acts as a vehicle, carrier or medium for the active ingredient.

The pharmaceutical composition may be formulated for immediate release or sustained release. A “sustained release formulation” is a formulation which is designed to slowly release a therapeutic agent in the body over an extended period of time, whereas an “immediate release formulation” is an formulation which is designed to quickly release a therapeutic agent in the body over a shortened period of time. In some cases the immediate release formulation may be coated such that the therapeutic agent is only released once it reached the desired target in the body (e.g. the stomach). In a specific embodiment, the pharmaceutical composition is formulated for immediate release.

The pharmaceutical composition may further comprise pharmaceutical excipients such as diluents, binders, fillers, glidants, disintegrants, lubricants, solubilizers, and combinations thereof Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises lactose monohydrate in an amount from about 10 to about 50% w/w, or from about 10 to about 40% w/w, or from about 10 to about 30% w/w, or from about 25 to about 35% w/w. In specific embodiments, the lactose monohydrate is present at about 10% w/w, at about 15% w/w, at about 20% w/w, at about 25% w/w, or at about 30% w/w. In a further specific embodiment, the lactose monohydrate is in an amount of about 16.5% w/w or about 27.5% w/w.

In further embodiments, the pharmaceutical composition comprises microcrystalline cellulose in an amount from about 10 to about 50% w/w, or from about 10 to about 45% w/w, or from about 10 to about 30% w/w, or from about 10 to about 25% w/w, or from about 10 to about 20% w/w, or from about 20 to about 40% w/w, or from about 25 to about 35% w/w. In specific embodiments, the microcrystalline cellulose is present in an amount of about 10%, or about 15%, or about 20%, or about 25%, or about 30%, or about 34%, or about 35%, or about 40%, or about 45%, or about 50% w/w. In a further specific embodiment, the microcrystalline cellulose is in an amount of about 18% w/w.

In further embodiments, the pharmaceutical composition comprises croscarmellose sodium in an amount from about 1 to about 20% w/w, or from about 1 to about 15% w/w, or from about 1 to about 10% w/w, or from about 1 to about 8% w/w, or from about 2 to about 8% w/w. In specific embodiments, the croscarmellose sodium is present in an amount of about 1%, or about 3%, or about 5%, or about 8%, or about 10%, or about 13%, or about 15% w/w. In a further specific embodiment, the croscarmellose sodium is in an amount of about 8.3% w/w.

In further embodiments, the pharmaceutical composition comprises colloidal silicon dioxide in an amount from about 0.5 to about 5% w/w, or from about 0.5 to about 4.5% w/w, or from about 0.5 to about 4% w/w, or from about 1.0 to about 2.0% w/w. In specific embodiments, the colloidal silicon dioxide is present in an amount of about 0.5% w/w, 0.75% w/w, 1% w/w, 1.25% w/w, 1.5% w/w, or 2% w/w. In a further specific embodiment, the colloidal silicon dioxide is present in an amount of about 1.7% w/w.

In further embodiments, the pharmaceutical composition comprises magnesium stearate in an amount from about 0.1 to about 3% w/w, or from about 0.1 to about 2.5% w/w, or from about 0.5 to about 3% w/w, or from about 0.5 to about 2.5% w/w, or from about 0.5 to about 2% w/w, or from about 0.5% to about 1.5% w/w, or from about 2 to about 3% w/w. In specific embodiments, the magnesium stearate is present in an amount of about 0.5%, or about 1%, or about 2%, or about 2.5%, or about 3% w/w. In a further specific embodiment, the magnesium stearate is in an amount of about 1.5% w/w.

In one embodiment, the pharmaceutical composition comprises:

a) about 15 to about 30% w/w of a solid dispersion comprising substantially amorphous ledipasvir dispersed within a polymer matrix formed by a pharmaceutically acceptable polymer, wherein the weight ratio of ledipasvir to polymer is from about 2:1 to about 1:2,

b) about 10 to about 40% w/w lactose monohydrate,

c) about 10 to about 40% w/w microcrystalline cellulose,

d) about 1 to about 10% w/w croscarmellose sodium,

e) about 0.5 to about 5% w/w colloidal silicon dioxide, and

f) about 0.1 to about 10% w/w magnesium stearate.

In some embodiments, the compositions are formulated in a unit dosage or pharmaceutical dosage form. The term “unit dosage forms” or “pharmaceutical dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet or capsule). The compounds are generally administered in a pharmaceutically effective amount. In some embodiments, each dosage unit contains from 3 mg to 2 g of ledipasvir. In other embodiments, the pharmaceutical dosage form comprises from about 3 to about 360 mg, or about 10 to about 200 mg, or about 10 to about 50 mg, or about 20 to about 40 mg, or about 25 to about 35 mg, or about 40 to about 140 mg, or about 50 to about 130 mg, or about 60 to about 120 mg, or about 70 to about 110 mg, or about 80 to about 100 mg. In specific embodiments, the pharmaceutical dosage form comprises about 40, or about 45, or about 50, or about 55, or about 60, or about 70, or about 80, or about 100, or about 120, or about 140, or about 160, or about 180, or about 200, or about 220 mg of ledipasvir. In a further specific embodiment, the pharmaceutical dosage form comprises about 90 mg of ledipasvir. In yet a further specific embodiment, the pharmaceutical dosage form comprises about 30 mg of ledipasvir. It will be understood, however, that the amount of the compound actually administered usually will be determined by a physician, in the light of the relevant circumstances, including the condition to be treated, the chosen route of administration, the actual compound administered and its relative activity, the age, weight and response of the individual patient, the severity of the patient's symptoms, and the like.

In one embodiment, the pharmaceutical composition, or alternatively, the pharmaceutical dosage form comprises about 90 mg of ledipasvir formulated in a solid dispersion comprising a polymer:ledipasvir ratio of 1:1, and wherein the solid dispersion is in an amount of about 30% w/w, lactose monohydrate in an amount from about 10 to about 40% w/w, microcrystalline cellulose in an amount from about 10 to about 40% w/w, croscarmellose sodium in an amount from about 1 to about 10% w/w, colloidal silicon dioxide in an amount from about 0.5 to about 5.0% w/w, and magnesium stearate in an amount from about 0.1 to about 10% w/w.

In another embodiment, the pharmaceutical composition, or alternatively, the pharmaceutical dosage form comprises about 30 mg of ledipasvir formulated in a solid dispersion comprising a polymer:ledipasvir ratio of 1:1, and wherein the solid dispersion is in an amount of about 30% w/w, lactose monohydrate in an amount from about 10 to about 40% w/w, microcrystalline cellulose in an amount from about 10 to about 40% w/w, croscarmellose sodium in an amount from about 1 to about 10% w/w, colloidal silicon dioxide in an amount from about 0.5 to about 5.0% w/w, and magnesium stearate in an amount from about 0.1 to about 10% w/w.

The tablets or pills of the present disclosure may be coated or otherwise compounded to provide a dosage form affording the advantage of prolonged action or to protect from the acid conditions of the stomach. The tablets may also be formulated for immediate release as previously described. In certain embodiments, the tablet comprises a film coating. A film coating of ledipasvir solid dispersions is useful for limiting photolytic degradation. Suitable film coatings are selected by routine screening of commercially available preparations. In one embodiment, the film coating is a polyvinylalcohol-based coating.

In one embodiment, the tablet comprises a) about 10 to about 40% w/w of the solid dispersion of ledipasvir; b) about 10 to about 40% w/w lactose monohydrate, c) about 10 to about 40% w/w microcrystalline cellulose, d) about 1 to about 10% w/w croscarmellose sodium, e) about 0.5 to about 5.0% w/w colloidal silicon dioxide, f) about 0.1 to about 10% w/w magnesium stearate, and g) optionally a film coating.

### 4. Methods of Making Solid Dispersions of Ledipasvir

Also provided are methods of making a solid dispersion comprising ledipasvir. Various techniques are well known in the art for preparing solid dispersions including, but not limited to melt-extrusion, spray-drying, lyophilization, and solution-evaporation.

Melt-extrusion is the process of embedding a compound in a thermoplastic carrier. The mixture is processed at elevated temperatures and pressures, which disperses the compound in the matrix at a molecular level to form a solid solution. Extruded material can be further processed into a variety of dosage forms, including capsules, tablets and transmucosal systems.

For the solution-evaporation method, the solid dispersion can be prepared by dissolving the compound in a suitable liquid solvent and then incorporating the solution directly into the melt of a polymer, which is then evaporated until a clear, solvent free film is left, The film is further dried to constant weight.

For the lyophilization technique, the compound and carrier can be co-dissolved in a common solvent, frozen and sublimed to obtain a lyophilized molecular dispersion.

For spray dried solid dispersions, the solid dispersion can be made by a) mixing the compound and polymer in a solvent to provide a feeder solution; and b) spray drying the feeder solution to provide the solid dispersion.

Spray dried solid dispersions of ledipasvir provided improved in vivo and in vitro performance and manufacturability/scalability, such as improved dissolution rate/solubility and stability, relative to the other formulation approaches, such as wet and dry granulation formulations. ledipasvir can be provided either as the free base, D-tartrate salt, crystalline acetone solvate, or other solvate as described herein.

The selection of the polymer for the solid dispersion is based on the stability and physical characteristics of the compound in the solution. Hypromellose and copovidone solid dispersions both showed adequate stability and physical characteristics. Accordingly, in one embodiment, the polymer used in the solid dispersion is selected from hypromellose and copovidone. Furthermore, the copovidone-based dispersion increased bioavailability more than the equivalent hypromellose-based formulation (F=30% and 22%, respectively) when spray dried at 2:1 API:polymer ratio. Bioavailability of the copovidone-based formulation was further enhanced by increasing the fraction of polymer to a 1:1 ratio, resulting in a bioavailability of 35% in famotidine pretreated dogs. Specific embodiments of the invention provide for a 2:1 ledipasvir:polymer ratio when making the solid dispersion. A further embodiment provides for a 1:1 ledipasvir:polymer ratio when making the solid dispersion. In another specific embodiment, the polymer used is copovidone. The use of copovidone at a 1:1 ledipasvir:polymer ratio provided for improved bioavailability with lower variability. These results are shown in Table 5 in Example 4.

After the compound is mixed with the polymer, the mixture can then be solubilized in a solvent. It is within the skill of those in the art to select an appropriate solvent based on the drug and/or polymer properties such as solubility, glass transition temperature, viscosity, and molecular weight. Acceptable solvents include, but are not limited to water, acetone, methyl acetate, ethyl acetate, chlorinated solvents, ethanol, dichloromethane, and methanol. In one embodiment, the solvent is selected from the group consisting of ethanol, dichloromethane, and methanol. In a further embodiment, the solvent is ethanol or methanol. In a specific embodiment, the solvent is ethanol.

Upon solubilization of the compound and polymer mixture with the solvent, the mixture may then be spray dried. Spray drying is a well-known process wherein a liquid feedstock is dispersed into droplets into a drying chamber along with a heated process gas stream to aid in solvent removal and to produce a powder product. Suitable spray drying parameters are known in the art, and it is within the knowledge of a skilled artisan in the field to select appropriate parameters for spray drying. The target feed concentration is generally about 10 to about 50% with a target of about 20% and a viscosity of about 15 to about 80 centipoise (cP). The inlet temperature of the spray dry apparatus is typically about 50-190° C., while the outlet temperature is about 30-90° C. The two fluid nozzle and hydraulic pressure nozzle can be used to spray dry compound I. The two fluid nozzle gas flow can be about 1-10 kg/hr, the hydraulic pressure nozzle flow can be about 15-300 kg/hr, and the chamber gas flow may be about 25-2500 kg/hr. The spray-dried material typically has particle size (D₉₀) under 80 μm. In some instances, a milling step may be used, if desired to further reduce the particle size. Further descriptions of spray drying methods and other techniques for forming amorphous dispersions are provided in U.S. Pat. No. 6,763,607 and U.S. Patent Publication No. 2006-0189633, the entirety of each of which is incorporated herein by reference.

Spray drying out of ethanol resulted in high yields (88, 90, 92%) across a wide range of spray-drying outlet temperatures (30-90° C.) with no material accumulation on the spray dry chamber, and the yields obtained from spray drying out of DCM were 60%, 78%, and 44%. Furthermore, ledipasvir demonstrated good chemical stability in the ethanolic feed solution.

### 5. Methods of Use

The solid dispersions, pharmaceutical compositions, pharmaceutical dosage forms, and tablets of ledipasvir described herein are administered to a patient suffering from hepatitis C virus (HCV) in a daily dose by oral administration. In one embodiment, the patient is human.

In one embodiment, the daily dose is 90 mg or 30 mg administered in the form of a tablet. In a related embodiment, the tablet comprises a) about 10 to about 40% w/w of the solid dispersion of ledipasvir; b) about 10 to about 40% w/w lactose monohydrate, c) about 10 to about 40% w/w microcrystalline cellulose, d) about 1 to about 10% w/w croscarmellose sodium, e) about 0.5 to about 5.0% w/w colloidal silicon dioxide, f) about 0.1 to about 10% w/w magnesium stearate, and g) optionally a film coating.

In one embodiment, the solid dispersions, pharmaceutical compositions, pharmaceutical dosage forms, and tablets of ledipasvir as described herein are effective in treating one or more of genotype 1 HCV infected subjects, genotype 2 HCV infected subjects, genotype 3 HCV infected subjects, genotype 4 HCV infected subjects, genotype 5 HCV infected subjects, and/or genotype 6 HCV infected subjects. In one embodiment, the solid dispersions, pharmaceutical compositions, pharmaceutical dosage forms, and tablets of ledipasvir as described herein are effective in treating genotype 1 HCV infected subjects, including genotype 1a and/or genotype 1b. In another embodiment, the solid dispersions, pharmaceutical compositions, pharmaceutical dosage forms, and tablets of ledipasvir as described herein are effective in treating genotype 2 HCV infected subjects, including genotype 2a, genotype 2b, genotype 2c and/or genotype 2d. In another embodiment, the solid dispersions, pharmaceutical compositions, pharmaceutical dosage forms, and tablets of ledipasvir as described herein are effective in treating genotype 3 HCV infected subjects, including genotype 3a, genotype 3b, genotype 3c, genotype 3d, genotype 3e and/or genotype 3f. In another embodiment, the solid dispersions, pharmaceutical compositions, pharmaceutical dosage forms, and tablets of ledipasvir as described herein are effective in treating genotype 4 HCV infected subjects, including genotype 4a, genotype 4b, genotype 4c, genotype 4d, genotype 4e, genotype 4f, genotype 4g, genotype 4h, genotype 4i and/or genotype 4j. In another embodiment, the solid dispersions, pharmaceutical compositions, pharmaceutical dosage forms, and tablets of ledipasvir as described herein are effective in treating genotype 5 HCV infected subjects, including genotype 5a. In another embodiment, the solid dispersions, pharmaceutical compositions, pharmaceutical dosage forms, and tablets of ledipasvir as described herein are effective in treating genotype 6 HCV infected subjects, including genotype 6a.

In some embodiments, the solid dispersions, pharmaceutical compositions, pharmaceutical dosage forms, and tablets of ledipasvir as described herein are administered, either alone or in combination with one or more therapeutic agent(s) for treating HCV (such as a HCV NS3 protease inhibitor and/or an inhibitor of HCV NS5B polymerase), for about 24 weeks, for about 16 weeks, or for about 12 weeks or less. In further embodiments, the solid dispersions, pharmaceutical compositions, pharmaceutical dosage forms, and tablets of ledipasvir are administered, either alone or in combination with one or more therapeutic agent(s) for treating HCV (such as a HCV NS3 protease inhibitor or an inhibitor of HCV NS5B polymerase), for about 24 weeks or less, about 22 weeks or less, about 20 weeks or less, about 18 weeks or less, about 16 weeks or less, about 12 weeks or less, about 10 weeks or less, about 8 weeks or less, about 6 weeks or less, or about 4 weeks or less. The solid dispersions, pharmaceutical compositions, pharmaceutical dosage forms, and tablets may be administered once daily, twice daily, once every other day, two times a week, three times a week, four times a week, or five times a week.

In further embodiments, a sustained virologic response is achieved at about 24 weeks, at about 20 weeks, at about 16 weeks, 12 weeks, at about 10 weeks, at about 8 weeks, at about 6 weeks, or at about 4 weeks, or at about 4 months, or at about 5 months, or at about 6 months, or at about 1 year, or at about 2 years.

### EXAMPLES

In the following examples and throughout this disclosure, abbreviations as used herein have respective meanings as follows:

| | |
|---|---|
| API | Active Pharmaceutical Ingredient |
| AUC | Area Under the Curve |
| BT | Breakthrough Rate |
| ° C. | Degrees Celsius |
| Cₘₐₓ | Maximum Concentration |
| cP | Centipoise |
| DCM | Dichloromethane |
| DSC | Differential Scanning Calorimetry |
| F | Bioavailability |
| g | Gram |
| GLSM | Geometric Least Squares Mean |
| h or hr | Hour |
| HCV | Hepatitis C virus |
| HPLC | High-performance Liquid Chromatography |
| ICH | International Conference on Harmonisation; |
| | Impurities guidelines |
| kg | Kilogram |
| L | Liter |
| LLOQ | Lower Limit of Quantification |
| mg | Milligram |
| min | Minute |
| mL | Milliliter |
| m | Meter |
| mm | Millimeter |
| PK | Pharmacokinetics |
| PS | Particle Size |
| RH | Relative Humidity |
| RNA | Ribonucleic Acid |
| SVR | Sustained Virologic Response |
| TGA | Thermogravimetric Analysis |
| vRVR | Very Rapid Viral Response |
| w | Weight |
| XRPD | X-ray Powder Diffraction |
| μm | Micrometer |
| μL | Microliter |
| SS-NMR | Solid-State Nuclear magnetic resonance |
| LOD | Loss on Drying |
| s | Second |
| imp | Impurity |
| deg | Degradation |
| PD | Pharmacodynamic |
| RSD | Relative Standard Deviation |
| nM | Nanomolar |
| HPMC | Hydroxypropyl methylcellulose |
| ng | Nanogram |
| CL/F | Apparent clearance |
| t_{1/2} | Half life |
| Vz/F | Apparent volume of distribution |
| Tₘₐₓ | Time to peak concentration |
| CI | Confidence interval |
| GMR | Geometric mean ratios |
| Cₗₐₛₜ | Last measure concentration |

### Example 1

### Synthesis of Amorphous Ledipasvir

Methods for making various forms of ledipasvir may be found in United States Patent Publication Nos. 2013/0324740 and 2013/0324496. Both of which are incorporated herein by reference. Following is a method for isolating amorphous free base of ledipasvir.

Combine ledipasvir acetone solvate (191.4 g) and acetonitrile (1356 g) in a reaction vessel and mix contents until a solution is achieved. Add this ledipasvir in acetonitrile solution slowly to another reaction vessel containing vigorously agitated water (7870 g). Agitate contents at about 23° C. for about 30 minutes. Filter the contents and dry at about 40-45° C. until constant weight is achieved to afford ledipasvir amorphous solid (146.4 g, 82% yield).

### Example 2

### Amorphous Solid Dispersion of Ledipasvir

To make the solid dispersion of ledipasvir, either the acetone solvate, D-tartrate salt, or amorphous free base of ledipasvir can be used. Other solvates of ledipasvir as described herein may also be used. Because of the high solubility in organic solvents and excipients and the ability to isolate the ledipasvir free base crystalline acetone solvate, this form was used in the amorphous solid dispersion of ledipasvir.

The spray dried solid dispersion approach achieved the most desirable characteristics relative to the other formulation approaches, which included: improved in vivo and in vitro performance and manufacturability/scalability.

The spray dry feed solution was prepared by solubilizing ledipasvir acetone solvate and polymer in the feed solvent. Aggressive mixing or homogenization was used to avoid clumping of the composition.

Different polymers were tested for preferred characteristics in the solid dispersions. Non-ionic such as hypromellose and copovidone solid dispersions both showed adequate stability and physical characteristics.

The feed solution was initially evaluated for appropriate solvent with regard to solubility, stability, and viscosity. Ethanol, methanol, and dichloromethane (DCM) all demonstrated excellent solubility (ledipasvir solubility >500 mg/mL). Ethanolic and DCM-based feed stocks were assessed for preparation ease and spray dried at a range of inlet and outlet temperatures to assess the robustness of the spray dry process. Both solvents gave rapid dissolution of ledipasvir and copovidone.

Spray drying out of ethanol resulted in high yields (88, 90, 92%) across a wide range of spray-drying outlet temperatures (49-70° C.) with no material accumulation on the spray dry chamber. Spray drying out of DCM resulted in yields of 60%, 78%, and 44%. Overall, the ledipasvir Solid Dispersion (50% w/w) in a ledipasvir to copovidone ratio of 1:1 demonstrated good chemical stability in the ethanolic feed solution.

An ethanolic solution of 10% ledipasvir acetone solvate and 10% copovidone was prepared using homogenization. Viscosity of ethanolic solutions of ledipasvir:copovidone were low, measured through 30% solids content (˜65 cP).

Spray drying was conducted using an Anhydro MS35 spray dryer. Table 1 presents the spray dry process parameters evaluated at 100 g-4000 g of total feed solution. Particle size data suggested sufficiently large particle size (10-14 μm mean PS) and was minimally affected by using higher spray rates or a larger diameter spray nozzle. Nozzle gas flow was not modulated to increase particle size.

TABLE 1
Ledipasvir Spray Dry Parameters on Anhydro MS35 Spray Dryer

| Parameter | Trial 1 | Trial 2 | Trial 3 | Trial 4 |
|---|---|---|---|---|
| Batch Size (g) | 100 | 250 | 250 | 4000 |
| Solids % | 20 | 20 | 20 | 20 |
| Feed Rate (mL/min) | 30 | 40 | 40 | 40 |
| Spray Nozzle (mm) | 1.0 | 1.0 | 1.2 | 1.2 |
| Nozzle Gas Flow | 6.0 | 6.0 | 6.0 | 6.0 |
| (kg/hr) | | | | |
| Chamber Gas Flow | 35.0 | 35.0 | 35.0 | 35.0 |
| (kg/hr) | | | | |
| Inlet Temp (° C.) | 125 | 165 | 165 | 165 |
| Outlet Temp (° C.) | 70 | 73 | 72 | 76 |
| PS d₁₀/d₅₀/d₉₀/mean | 4/9/18/10 | 5/10/20/12 | 5/10/19/11 | 6/12/22/14 |
| (μm) | | | | |
| Post Spray LOD (%) | 5.56 | 4.86 | 4.29 | 3.42 |

Organic volatile impurities, including the spray dry solvent ethanol and residual acetone from ledipasvir acetone solvate are rapidly removed during secondary drying in a tray oven 60° C., purged with room air. Loss on drying (LOD) was proportionately slower and is attributable to water, which was later confirmed by Karl Fischer titration.

Residual ethanol was reduced below ICH guidelines of 0.5% w/w by 6 hours of drying. Ethanol content upon completion of drying was 0.08% w/w, and residual acetone was 0.002%, indicating that the secondary drying process is adequate for removal of residual solvent.

### Example 3

### Tablet Preparation and Formulation

The following provides an example method for making tablets using the amorphous solid dispersions comprising ledipasvir. The amorphous solid dispersion comprising ledipasvir was blended with excipients and milled to facilitate mixing and blend uniformity. An in-process milling step was required to deagglomerate relatively small but hard agglomerates present in the drug substance. To limit any loss of drug substance, the ledipasvir drug substance was blended with all intragranular excipients prior to milling through a conical screen mill with a 024R screen and a tip speed of 23.7 m/s. No drug substance agglomerates were visually observed after milling. Blend uniformity was achieved after the milling step for the ledipasvir powder blend, 0.4% w/w. A secondary blend was conducted prior to lubrication, followed by roller compaction and milling through an in-line oscillating mill. Adequate uniformity was again demonstrated for the low strength final powder blend. This process results in powder blends with satisfactory flow characteristics and compression properties. The powder blend was blended with a lubricant prior to roller compaction and milling through as oscillating mill. The granules were then mixed with a lubricant prior to tablet compression. The total resulting core tablet weight 250 mg. The tablet weight was maintained for the different dosage strengths by offsetting lactose content as a function of ledipasvir content.

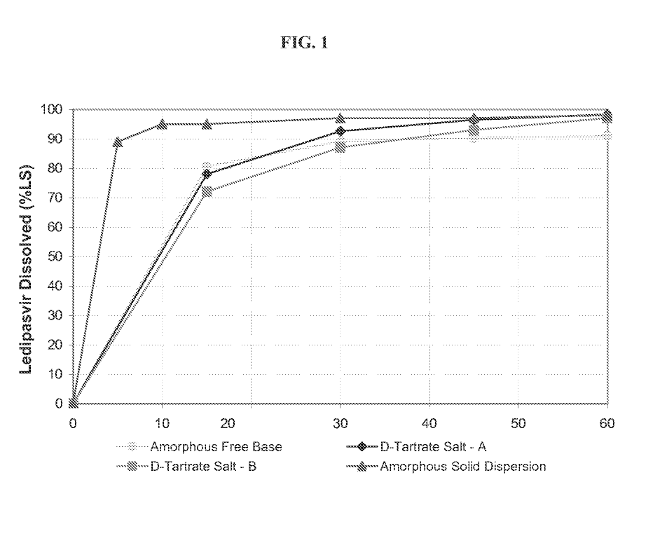

FIG. 1 displays the dissolution of the solid dispersion tablet compared to the dissolution of the conventional amorphous free base and D-tartrate salt tablets. Chemical stability of the solid dispersion tablet is shown in Table 2. As noted above, the amorphous solid dispersion tables showed improved dissolution over the amorphous free base conventional formulations and the crystalline salt conventional formulations.

TABLE 2
Stability of Ledipasvir Amorphous Solid Dispersion Tablets, 30 mg

| | | HPLC Assay | | |
|---|---|---|---|---|
| Time | | % | % imp/deg | Dissolution |
| point/condition | Appearance | Ledipasvir | products | (% at 45 min) |
| 25° C./75% RH | | | | |
| 0 months | Conforms | 96.1 | 0.5 | |
| 1 month | Conforms | 95.8 | 0.5 | |
| 3 months | Conforms | 96.5 | 0.5 | |
| 6 months | Conforms | 95.8 | 0.4 | |
| 40° C./75% RH | | | | |
| 0 months | Conforms | 96.1 | 0.5 | 97 |
| 1 month | Conforms | 96.4 | 0.5 | |
| 2 months | Conforms | 95.4 | 0.5 | 97 |
| 3 months | Conforms | 96.1 | 0.4 | |
| 6 months | Conforms | 95.8 | 0.5 | 97 |

Film-coating of ledipasvir amorphous solid dispersion tablets is provided to reduce photolytic degradation. Tablets were coated to a target 5% weight gain. The film-coating material was changed to a polyvinylalcohol-based coating. Exemplary tablet formulations are provided in Table 3.

TABLE 3
Composition of Tablets Comprising the Solid Dispersion of Ledipasvir

| | | Unit | Unit | | Unit |
|---|---|---|---|---|---|
| | | Formula | Formula | | Formula |
| | | (30 mg/ | (90 mg/ | | (90 mg/ |
| Ingredient | % w/w | tablet) | tablet) | % w/w | tablet) |
| Ledipasvir Solid | 30.00 | 60.0^{b} | 180.0^{a} | 30.00 | 180.0^{a} |
| Dispersion, 50% w/w | | | | | |
| Bulk Powder | | | | | |
| Lactose Monohydrate | 30.00 | 60.0 | 180.0 | 27.50 | 165.0 |
| Microcrystalline | 34.00 | 68.0 | 204.0 | 30.00 | 180.0 |
| Cellulose | | | | | |
| Croscarmellose Sodium | 5.00 | 10.0 | 30.0 | 8.30 | 50.0 |
| Magnesium Stearate | 1.00 | 2.0 | 6.0 | 2.50 | 15.00 |
| Colloidal Silicon Dioxide | — | — | — | 1.70 | 10.00 |
| Total Tablet Core | 100.0 | 200.0 | 600.0 | 100.0 | 600.0 |
| Weight | | | | | |
| Film coating | 5.00 | 10.0 | 30.0 | 5.00 | 30.0 |
| Purified Water | — | — | — | — | — |
| Total Coated Tablet | | 210.0 | 630.0 | | 630.0 |
| Weight | | | | | |

^{a}60 mg and 180 mg of ledipasvir Solid Dispersion, 50% w/w, are equivalent to 30 mg and 90 mg of ledipasvir free base, respectively.

### Example 4

### Bioavailability of Compositions Comprising Ledipasvir

To study the pharmacokinetics of ledipasvir, different formulations comprising either the amorphous free base or the crystalline D-tartrate salt both in conventional formulations, or the amorphous solid dispersion of ledipasvir using a variety of polymers in different ratios were made.

### 1. Dose Selection of Ledipasvir

The maximum median HCV RNA log 10 reduction was 3 or greater for all cohorts dosed with >3 mg of ledipasvir. An Eₘₐₓ PK/PD model indicates that the exposures achieved following administration of the 30 mg dose provides >95% of maximal antiviral response in genotype la HCV infected subjects. It was also observed that 30 mg or greater of ledipasvir likely provided coverage of some drug related mutations that doses less than 30 mg did not, based on an analysis of NS5A mutants that arose in response to exposure to ledipasvir. Therefore, 30 mg and 90 mg of ledipasvir were selected as the dose for the formulations described herein.

Further studies suggest that, when ledipasvir is administered in combination with other therapeutic agents, the breakthrough (BT) rate (number of patients with HCV RNA>lower limit of quantification (LLOQ) after having achieved vRVR/total number of patients who achieved vRVR), is higher with doses of 30 mg (BT=33%, 11/33; 30 mg ledipasvir), than with doses of 90 mg (BT=12%, 9/74; 90 mg ledipasvir). Therefore, the 90 mg dose of ledipasvir may confer a greater antiviral coverage that prevents viral breakthrough.

### 2. Bioavailability Studies

A series of in vivo experiments were conducted to evaluate the potential benefit of the solid dispersion approach relative to conventional formulations, as well as to optimize the solid dispersion by identifying the most beneficial polymer type and relative polymer concentration within the dispersion.

Equivalent bioavailability was achieved between formulations comprising the free base amorphous form (4% w/w, 10 mg amorphous free base tablet) and formulations comprising the D-tartrate salt of ledipasvir (5.85% w/w, 10 mg D-tartrate salt tablet) in the pentagastrin pretreated dog model, as shown in Table 4. Pentagastrin is a synthetic polypeptide that stimulates the secretion of gastric acid, pepsin, and intrinsic factor.

TABLE 4
Mean (RSD) Pharmacokinetic Parameters of Ledipasvir Following Oral
Administration of Tablets, 25 mg, in Beagle Dogs (n = 6)

| Drug | | | | |
|---|---|---|---|---|
| Substance | | Cₘₐₓ | AUC₀₋₂₄ | |
| Form | Pretreatment | (nM) | (nM * hr) | F (%) |
| Amorphous | Pentagastrin | 743 (17) | 8028 (22) | 71 |
| Free base | | | | |
| Crystalline | Pentagastrin | 665 (38) | 7623 (44) | 67 |
| D-tartrate | | | | |

Because these formulations displayed similar PK properties and the isolation properties of the D-tartrate salt were preferable to the free base amorphous form, the D-tartrate salt formulation was chosen to compare to the amorphous solid dispersion compositions. For these studies, 30 mg tablets comprising the D-tartrate salt of ledipasvir and 30 mg or 90 mg tablets comprising the amorphous solid dispersion of ledipasvir were used. Dog pharmacokinetic results for select immediate release ledipasvir tablets comprising ledipasvir solid dispersions are shown in Table 5.

TABLE 5
Mean (RSD) Pharmacokinetic Parameters of Ledipasvir after Oral
Administration of Ledipasvir Tablets Containing Ledipasvir
Spray-Dried Solid Dispersions to Fasted Beagle Dogs (n = 6)

| | Ledi- | | | | | |
|---|---|---|---|---|---|---|
| | pasvir: | | | | | |
| | polymer | Dose | Pre- | Cₘₐₓ | AUC₀₋₂₄ | F |
| Polymer | Ratio | (mg) | treatment | (nM) | (nM*hr) | (%) |
| D-tartrate | N/A | 30 | Pentagastrin | 665 (38) | 7623 (44) | 67 |
| Ledipasvir | | | Famotidine | 154 (44) | 1038 (41) | 9 |
| Tablets | | 90 | Pentagastrin | 1831 (28) | 18086 (36) | 54 |
| | | | Famotidine | 349 (37) | 3322 (40) | 10 |
| Amorphous | 2:1 | 30 | Famotidine | 251 (51) | 2553 (54) | 22 |
| Solid | | | | | | |
| Dispersion | | | | | | |
| Ledipasvir | | | | | | |
| Tablet: | | | | | | |
| HPMC | | | | | | |
| Amorphous | 2:1 | 30 | Famotidine | 369 (26) | 3383 (36) | 30 |
| Solid | 1:1 | | Pentagastrin | 983 (22) | 10541 (24) | 93 |
| Dispersion | 1:1 | | Famotidine | 393 (30) | 3930 (20) | 35 |
| Ledipasvir | 1:1 | 90 | Pentagastrin | 1644 (38) | 20908 (41) | 62 |
| Tablet: | 1:1 | | Famotidine | 740 (24) | 7722 (28) | 23 |
| Copovidone | | | | | | |

In pentagastrin pretreated animals, an approximate 40% increase in exposure and a 2-fold decrease in variability were noted. More importantly in famotidine pretreated animals, up to a 3.5-fold increase in bioavailability was observed compared to the D-tartrate salt tablet formulations.

A copovidone-based dispersion increased bioavailability more than the equivalent hypromellose-based formulation (F=30% and 22%, respectively) when spray dried at 2:1 API:polymer ratio. Bioavailability of the copovidone-based formulation was further enhanced by increasing the fraction of polymer to a 1:1 ratio, resulting in a bioavailability of 35% in famotidine pretreated dogs.

Because of the improved in vivo performance and acceptable stability and physical properties, a 1:1 mixture of ledipasvir:copovidone was chosen as the spray-dried material.

### 3. Conclusions

Formulations comprising the amorphous solid dispersions proved to be advantageous over formulations comprising either the amorphous free base or the D-tartrate salt. It was observed that the bioavailability of amorphous free base formulations was similar to D-tartrate salt formulations. Additional data showed a decrease in bioavailability when ledipasvir was dosed with gastric acid suppressing agents, indicating an unfavorable drug-drug interaction in free base amorphous and D-tartrate salt formulations of ledipasvir. A solid dispersion using spray drying with a hydrophilic polymer was identified to have acceptable stability, physical characteristics, and in vivo performance. A rapidly disintegrating tablet was developed using a dry granulation process and commonly used excipients. A bioavailability study comparing formulations comprising the D-tartrate salt with formulations comprising the amorphous solid dispersion showed improved biopharmaceutical performance and overcame much of the negative drug-drug interactions with acid suppressive therapies seen in the D-tartrate salt formulations.

### Example 5

### Reduction of Food Effect in Solid Dispersions of Ledipasvir

Conventional formulations of ledipasvir have been demonstrated to have a negative food effect. Table 6 summarizes PK parameters of ledipasvir following a single dose of ledipasvir, 30 mg, under fasted and fed conditions. The ledipasvir PK profile was altered in the presence of food. Specifically, the high-fat meal appeared to delay ledipasvir absorption, prolong Tₘₐₓ (median Tₘₐₓ of 8 hours), and decreased ledipasvir plasma exposure (approximately 45% decrease each in mean Cₘₐₓ, AUCₗₐₛₜ, and AUC_{inf}, respectively).

TABLE 6
Effect of Food on Plasma Ledipasvir PK Parameters Following
Single-dose Administration of a Conventional Formulation of Ledipasvir

| | Mean (% CV) | | | |
|---|---|---|---|---|
| | Ledipasvir | | Ledipasvir | |
| | 30 mg | | 30 mg Fed | |
| PK Parameter | (N = 8) | | (N = 8) | |
| Cₘₐₓ (ng/mL) | 73.1 | (50.8) | 36.5 | (22.6) |
| Tₘₐₓ (h) | 6.00 | (5.00, 6.00) | 8.00 | (7.00, 8.00) |
| AUCₗₐₛₜ | 1988.2 | (58.2) | 996.5 | (21.6) |
| (ng · h/mL) | | | | |
| AUC_{inf} | 2415.9 | (60.3) | 1175.0 | (25.3) |
| (ng · h/mL) | | | | |
| t_{1/2} (h) | 39.82 | (33.15, 41.65) | 36.83 | (22.19, 49.08) |
| CL/F (mL/h) | 17,034.5 | (58.6) | 26,917.9 | (23.6) |
| V_{z}/F (mL) | 876,546.3 | (44.2) | 1,386,469 | (24.9) |
| Cₗₐₛₜ (ng/mL) | 6.8 | (68.0) | 3.1 | (42.2) |

Table 7 presents the ratio of the GLSMs (conventional formulation of ledipasvir 30 mg under fasted conditions/ledipasvir 30 mg under fed conditions) for each of the primary PK parameters.

TABLE 7
Statistical Evaluations of Ledipasvir PK Parameters for Food Effect

| | Geometric Least Squares | | | |
|---|---|---|---|---|
| | Mean (GLSM) | | | |
| | Ledipasvir | Ledipasvir | GLSM Ratio | 90% |
| | 30 mg Fed | 30 mg Fasted | (Fed/Fasted) | Confidence |
| | (N = 8) | (N = 8) | % | Interval |
| Cₘₐₓ (ng/mL) | 35.87 | 65.33 | 54.90 | 39.10, 77.08 |
| AUCₗₐₛₜ | 977.76 | 1724.28 | 56.71 | 38.87, 82.73 |
| (ng · hr/mL) | | | | |
| AUC_{inf} | 1143.64 | 2058.78 | 55.55 | 36.88, 83.67 |
| (ng · hr/mL) | | | | |

Similar median half-lives of ledipasvir were observed independent of administration under fasted or fed conditions (t_{1/2} of 39.82 hours under fasted conditions vs 36.83 hours under fed conditions) indicating that food decreased the bioavailability of ledipasvir in a conventional formulation by reducing its solubility/dissolution rate and/or absorption.

Interestingly, the combination of ledipasvir formulated as a solid dispersion and sofosbuvir, another anti-HCV agent, lacked a negative food effect. These results are shown in Table 8.

Similar ledipasvir plasma exposures (AUC and Cₘₐₓ) were achieved upon administration of ledipasvir under fasted or fed conditions. The % GMR and associated 90% CIs (fed/fasted treatments) were within the equivalence bounds of 70-143%. As such, the combination of sofosbuvir and ledipasvir, as the solid dispersion, may be administered without regard to food. It is contemplated that the elimination of the food effect is at least partially attributable to formulating ledipasvir into the solid dispersion.

TABLE 8
Pharmacokinetic Data for Ledipasvir on Administration of Sofosbuvir/Ledipasvir
Solid Dispersion Tablets Fasted or with a Moderate-Fat Meal or with a High-Calorie/High
Fat Meal
Ledipasvir (n = 27)

| | Sofosbuvir/Ledipasvir | Sofosbuvir/Ledipasvir | |
|---|---|---|---|
| | Solid Dispersion | Solid Dispersion | % GMR (90% CI) |
| | Tablet | Tablet | Moderate- |
| Mean (% CV) | Fasted | Moderate-Fat Meal | Fat/Fasted |
| AUC_{inf} (ng · hr/mL) | 9610 (52.3) | 10100 (33.8) | 120 (103, 141) |
| AUCₗₐₛₜ (ng · hr/mL) | 7940 (51.0) | 8220 (30.0) | 118 (101, 139) |
| Cₘₐₓ (ng/mL) | 310 (45.4) | 313 (26.0) | 112 (96.0, 131) |
| | | Sofosbuvir/Ledipasvir | |
| | Sofosbuvir/Ledipasvir | Solid Dispersion | |
| | Solid Dispersion | Tablet | |
| | Tablet | High-Calorie/High- | GMR (90% CI) |
| | Fasted | Fat Meal | High-Fat/Fasted |
| AUC_{inf} (ng · hr/mL) | 9610 (52.3) | 8740 (34.0) | 107 (92.0, 126) |
| AUCₗₐₛₜ (ng · hr/mL) | 7940 (51.0) | 7350 (31.3) | 107 (91.0, 126) |
| Cₘₐₓ (ng/mL) | 310 (45.4) | 254 (27.5) | 92.0 (79.0, 108) |

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A solid dispersion comprising ledipasvir having the formula: 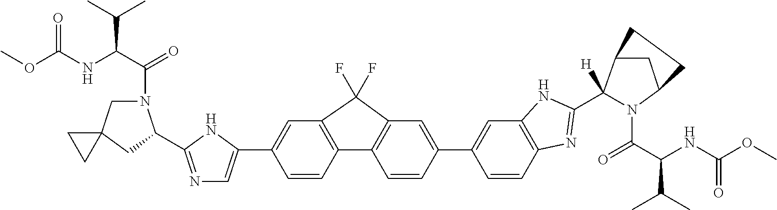
wherein ledipasvir is dispersed within a polymer matrix formed by a pharmaceutically acceptable polymer, and further wherein ledipasvir is substantially amorphous.

2. The solid dispersion of claim 1, wherein the polymer is hydrophilic.

3. The solid dispersion of claim 1, wherein the polymer is a non-ionic polymer.

4. The solid dispersion of claim 1, wherein the polymer is selected from the group consisting of hypromellose, copovidone, and povidone.

5. The solid dispersion of claim 4, wherein the polymer is copovidone.

6. The solid dispersion of claim 1, wherein the polymer is an ionic polymer.

7. The solid dispersion of claim 6, wherein the ionic polymer is selected from the group consisting of hydroxypropyl methylcellulose acetate-succinate, hydroxypropyl methylcellulose phthalate, and cellulose acetate phthalate.

8. The solid dispersion of claim 1, wherein the weight ratio of ledipasvir to polymer is from about 5:1 to about 1:5.

9. The solid dispersion of claim 8, wherein the weight ratio of ledipasvir to polymer is from about 2:1 to about 1:2.

10. The solid dispersion of claim 9, wherein the weight ratio of ledipasvir to polymer is about 1:1.

11. The solid dispersion of claim 9, wherein the weight ratio of ledipasvir to polymer is about 2:1.

12. A pharmaceutical composition comprising the solid dispersion of claim 1 and a pharmaceutically acceptable carrier.

13. The pharmaceutical composition of claim 12, comprising from about 5% to about 75% w/w of the solid dispersion.

14. The pharmaceutical composition of claim 12, comprising from about 20% to about 40% w/w of the solid dispersion.

15. The pharmaceutical composition of claim 12, wherein the composition is formulated for immediate release.

16. The pharmaceutical composition of claim 12, further comprising one or more of a diluent, a disintegrant, a glidant, a lubricant, and any combination thereof.

17. The pharmaceutical composition of claim 16, wherein the diluent is lactose monohydrate and is present in an amount from about 10 to about 30% w/w.

18. The pharmaceutical composition of claim 16, wherein the disintegrant is microcrystalline cellulose and is present in an amount from about 10 to about 40% w/w.

19. The pharmaceutical composition of claim 16, wherein the disintegrant is croscarmellose sodium and is present in an amount from about 1 to about 10% w/w.

20. The pharmaceutical composition of claim 16, wherein the glidant is colloidal silicon dioxide and is present in an amount from about 0.5 to about 5% w/w

21. The pharmaceutical composition of claim 16, wherein the lubricant is magnesium stearate and is present in an amount from about 0.1 to about 10% w/w.

22. The pharmaceutical composition of claim 12, comprising about 30% w/w of the solid dispersion.

23. The pharmaceutical composition of claim 22, further comprising
a) about 10 to about 40% w/w lactose monohydrate,
b) about 10 to about 40% w/w microcrystalline cellulose,
c) about 1 to about 10% w/w croscarmellose sodium,
d) about 0.5 to about 5% w/w colloidal silicon dioxide, and
e) about 0.1 to about 10% w/w magnesium stearate.

24. A pharmaceutical dosage form comprising the pharmaceutical composition of claim 12, wherein the dosage form comprises from about 3 to about 360 mg of the compound.

25. The pharmaceutical dosage form of claim 24, wherein the dosage form comprises from about 10 to about 100 mg of the compound.

26. The pharmaceutical dosage form of claim 25, wherein the dosage form comprises about 90 mg of the compound.

27. The pharmaceutical dosage form of claim 25, wherein the dosage form comprises about 30 mg of the compound.

28. A tablet comprising the pharmaceutical dosage form of claim 24.

29. The tablet of claim 28, further comprising a film coating.

30. The tablet of claim 29, wherein the film coating is a polyvinylalcohol-based coating.

31. The tablet of claim 28, comprising about 10 to about 40% w/w of the solid dispersion.

32. The tablet of claim 31, comprising about 30% w/w of the solid dispersion.

33. The tablet of claim 28, comprising about 50 to about 130 mg of ledipasvir.

34. The tablet of claim 33, comprising about 90 mg of ledipasvir.

35. The tablet of claim 33, comprising about 30 mg of ledipasvir.

36. The tablet of claim 31 further comprising:
a) about 10 to about 40% w/w lactose monohydrate,
b) about 10 to about 40% w/w microcrystalline cellulose,
c) about 1 to about 10% w/w croscarmellose sodium,
d) about 0.5 to about 5% w/w colloidal silicon dioxide, and
e) about 0.1 to about 10% w/w magnesium stearate.

37. The tablet of claim 36 further comprising a film coating.

38. A method of treating hepatitis C in a human patient in need thereof comprising administering to the patient a therapeutically effective amount of the solid dispersion of claim 1.

39. A method of making a solid dispersion of claim 1 comprising:
a) mixing ledipasvir and polymer in a solvent to provide a feeder solution;
b) spray drying the feeder solution to provide the solid dispersion.

40. The method of claim 39, wherein ledipasvir is provided as either the free base, salt, or solvate.

41. The method of claim 39, wherein the solvent is selected from ethanol, methanol, or dichloromethane.

42. The method of claim 41, wherein the solvent is ethanol.
